(19) United States
(12) Patent Application Publication
(10) Pub. No.: US 2022/0071491 A1
(43) Pub. Date: Mar. 10, 2022

(54) LIGHT IRRADIATION DEVICE

(71) Applicant: SEOUL VIOSYS CO., LTD., Gyeonggi-do (KR)

(72) Inventors: Hee Ho BAE, Gyeonggi-do (KR); Yeong Min YOON, Gyeonggi-do (KR); A Young LEE, Gyeonggi-do (KR)

(73) Assignee: SEOUL VIOSYS CO., LTD., Gyeonggi-do (KR)

(21) Appl. No.: 17/478,686

(22) Filed: Sep. 17, 2021

Related U.S. Application Data
(63) Continuation of application No. PCT/KR2020/003899, filed on Mar. 20, 2020.
(60) Provisional application No. 62/821,611, filed on Mar. 21, 2019.

(51) Int. Cl. A61B 5/00; G01N 21/33; G01N 21/31

(52) U.S. Cl. CPC A61B 5/0059; G01N 21/314; G01N 21/33; A61B 5/411

(57) ABSTRACT

A light irradiation device present disclosure includes at least one first light source that emits first light in a wavelength band for treating skin, an erythema detector that obtains color information of the skin to detect whether erythema of the skin is generated by irradiation of the first light, and a control unit that determines whether erythema is generated on the basis of the color information and controls driving of the first light source according to whether erythema is generated. The erythema detector includes at least one second light source that emits second light in a visible light wavelength band to the skin, and at least one sensor that receives the second light traveling through the skin.

### CROSS-REFERENCE OF RELATED APPLICATIONS AND PRIORITY

The Present application is a continuation of International Application No. PCT/KR2020/003899 filed Mar. 20, 2020 which claims priority to and benefit of U.S. Provisional Application No. 62/821,611 filed Mar. 21, 2019, the disclosure of which are incorporated by reference as if fully set forth herein by their entirety.

### TECHNICAL FIELD

The following description relates to a light irradiation device.

### BACKGROUND

Recently, various treatment devices using an ultraviolet light have been developed. In general, the ultraviolet light is known to have a bactericidal effect, and a conventional ultraviolet light therapy device is used by a method in which a conventional ultraviolet lamp is operated near a skin to irradiate the ultraviolet light to an area requiring treatment.

A light such as the ultraviolet light, exposed to the skin, may lead to a skin abnormality such as erythema. The skin abnormality such as erythema may not be confirmed in real time.

### SUMMARY

The object of the present disclosure provides a phototherapy device secured safety.

A light irradiation device according to an example embodiment of the present disclosure includes at least one first light source that emits a first light of a wavelength band for treating a skin, an erythema detector that obtains color information of the skin to detect whether erythema of the skin occurs due to irradiation of the first light, and a controller that determines whether erythema is generated based on the color information and control driving of the first light source depending on whether erythema is generated. The erythema detector includes at least one second light source that emits a second light of a visible light wavelength band to the skin, and at least one sensor that receives the second light traveling through the skin.

In an example embodiment of the present disclosure, the second light may be a light corresponding to a wavelength band of a visible light.

In an example embodiment of the present disclosure, the sensor may detect the second light reflected, scattered, or dispersed by the skin.

In an example embodiment of the present disclosure, the controller may include a comparator which derives and compares a change rate between a skin color of the skin detected before the first light is applied and a skin color of the skin detected after the first light is applied to determine whether erythema occurs or not.

In an example embodiment of the present disclosure, the skin color may be represented by color coordinate values in a CIE LAB color space.

In an example embodiment of the present disclosure, the controller may pre-set virtual skin color measurement sheets depending on a type of external lighting, may additionally correct a value due to a difference in the skin color measurement sheets depending on the type of external lighting, and then may derive and compare the change rate between the skin color of the skin detected before the first light is applied and the skin color of the skin detected after the first light is applied, to determine whether erythema occurs or not.

In an example embodiment of the present disclosure, the controller may include the comparator comparing the change rate between a predetermined skin color and a skin color of the skin detected from the sensor to determine whether erythema occurs or not.

In an example embodiment of the present disclosure, the sensor may include a CCD, a CMOS image sensor, or a photodiode.

In an example embodiment of the present disclosure, the erythema detector may further include a temperature sensor which measures a temperature of the skin.

In an example embodiment of the present disclosure, the temperature sensor may include an infrared sensor or a contact sensor which is directly contact with the skin to measure the temperature.

In an example embodiment of the present disclosure, the light irradiation device may further include a main body on which the first light source and the erythema detector are mounted, and the main body may have flexibility

In an example embodiment of the present disclosure, the first light may be applied to a first region of the skin and at least one of the second light source or the sensor may be movable in the first region.

In an example embodiment of the present disclosure, the main body may include a rail provided on a surface facing the skin and along a movement path of at least one of the second light source and the sensor.

In an example embodiment of the present disclosure, the first light may be a light of a blue wavelength band.

In an example embodiment of the present disclosure, the first light may be a light of a red to infrared wavelength band.

In an example embodiment of the present disclosure, the first light may be a light of an ultraviolet wavelength band.

In an example embodiment of the present disclosure, the first light may be a light in which at least two wavelength bands of ultraviolet, visible and infrared wavelength bands are combined.

In an example embodiment of the present disclosure, the second light source may have a wavelength band of about 380 nm to about 780 nm, may have an area of about 55% or more of an area of a normalized solar spectrum within a range of about 2,600K to about 7,000K, and normalized solar spectrum may be represented by the following Equation 1.

$$E ( λ , T ) = \frac{2 h {c}^{2}}{{λ}^{5}} \cdot \frac{1}{{e}^{hc / λ kT} - 1} \qquad Equation 1$$

where λ: wavelength (um); h: Planck's constant;
c: speed of light;
T: absolute temperature; and
k: Boltzmann constant

In an example embodiment of the present disclosure, a light irradiation device includes at least one first light source configured to emit first light of a wavelength band for treating a target skin, an erythema detector configured to obtain color information of the target skin, and a controller configured to: determine whether erythema of the target skin has occurred based on the color information and control driving of the first light source depending on occurrence of erythema. The erythema detector further includes at least one second light source configured to emit second light to the target skin, and at least one sensor configured to receive the second light passing through the target skin and provide the color information of the target skin.

In another variant, the sensor detects the second light reflected, scattered, or dispersed by the target skin.

In another variant, the controller further includes a comparator which compares between a skin color of the target skin detected before the first light is applied and a skin color of the target skin detected after the first light is applied and derives a change rate in the skin color of the target skin, the change rate indicative of whether erythema has occurred or not.

In another variant, the controller is further configured to pre-set virtual skin color measurement sheets depending on a type of external lighting, additionally correct a value due to a difference in the skin color measurement sheets due to the difference in the type of external lighting, and then derive and compare the change rate between the skin color of the skin detected before the first light is applied and the skin color of the skin detected after the first light is applied, to determine whether erythema occurs or not.

An example embodiment of the present disclosure provides a high safety light irradiation device.

### DESCRIPTION OF DRAWINGS

FIG. 1 is a plan view illustrating a light irradiation device according to an example embodiment of the present disclosure.

FIG. 2 is a block diagram illustrating the light irradiation device of FIG. 1.

FIG. 3A is a flowchart illustrating an operation sequence of a light irradiation device according to an example embodiment of the present disclosure.

FIG. 3B is a flowchart illustrating an operation sequence of a light irradiation device according to another example embodiment of the present disclosure.

FIG. 4A is a perspective view of a light irradiation device according to an example embodiment of the present disclosure, which illustrates a light irradiation device manufactured in a form of a mask.

FIG. 4B is a plan view illustrating a surface facing a face, that is, a back surface of the light irradiation device, in the light irradiation device of FIG. 4A.

FIG. 4C is a side view in which the light irradiation device of FIG. 4A is worn on the face.

FIGS. 5A to 5D illustrates an erythema detector which is movable within a light irradiation device, where:

FIG. 5A shows one exemplary position of the erythema detector;

FIG. 5B shows another exemplary position of the erythema detector;

FIG. 5C shows another exemplary position of the erythema detector; and

FIG. 5D shows another exemplary position of the erythema detector.

FIG. 6A is a perspective view of a light irradiation device according to an example embodiment of the present disclosure and illustrates a light irradiation device manufactured in a wearable form.

FIG. 6B is a perspective view illustrating a state in which the light irradiation device of FIG. 6A is worn on a human arm.

FIGS. 7A to 7C illustrate a minimum amount of erythema dose within a range in which erythema does not occur in a first light irradiation depending on a type of skin, where:

FIG. 7A illustrates a case where UVB is used as the first light;

FIG. 7B illustrates a case where UVA is used as the first light; and

FIG. 7C illustrates a case where a combination of UVA and UVB is used as the first light.

### DETAILED DESCRIPTION OF EMBODIMENTS

The inventive concept may be variously modified and may have various forms, and specific embodiments are illustrated in the drawings and described in detail in the disclosure. However, this is not intended to limit the inventive concept to the specific form disclosed and it should be understood to include all modifications, equivalents, and substitutes included in the spirit and scope of the inventive concept.

Hereinafter, a preferred embodiment of the inventive concept will be described in detail with reference to the accompanying drawings.

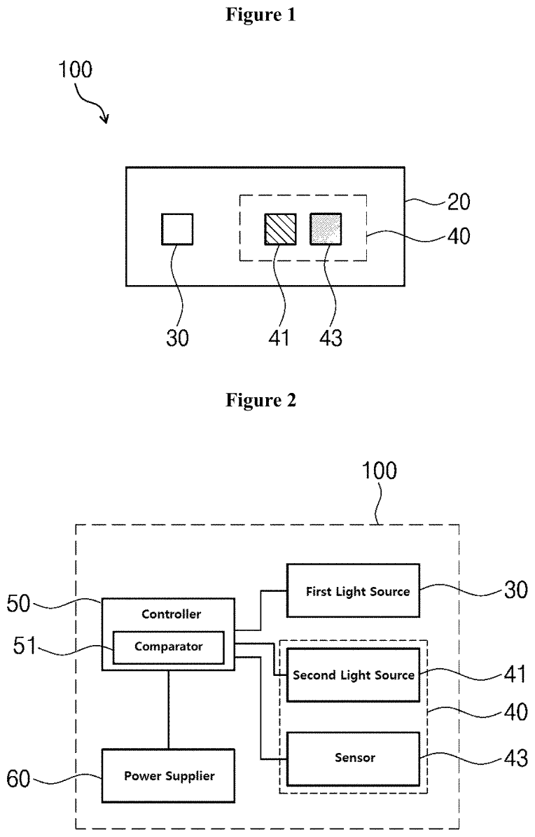

FIG. 1 is a plan view illustrating a light irradiation device 100 according to an example embodiment of the present disclosure, and FIG. 2 is a block diagram illustrating the light irradiation device 100 of FIG. 1.

Referring to FIGS. 1 and 2, the light irradiation device 100 according to an example embodiment of the present disclosure includes a substrate 20, a first light source 30 for emitting a first light for treating a skin, and an erythema detector 40 for obtaining color information of the skin to detect whether erythema of the skin occurs due to irradiation of the first light. The first light source 30 and the erythema detector 40 may be connected to a controller 50, which determines whether erythema is generated based on the color information and controls an operation of the first light source 30 depending whether erythema is generated, and a power supplier 60 may supply power to the controller 50, the first light source 30, a second light source 41, and a sensor 43.

The substrate 20 may not be particularly limited as long as it is capable of mounting the first light source 30 and the erythema detector 40, and may be provided in various forms. The substrate 20 may be provided in a form in which wires are included to supply power to the first light source 30 and the erythema detector 40. The substrate 20 may be formed of, for example, a metal substrate, a printed circuit board, or the like on which wires are formed.

In an example embodiment of the present disclosure, the skin corresponds to an object to be treated, which receives a light form the light irradiation device 100 according to an example embodiment of the present disclosure, and includes skins of other animals as well as a skin of a human. Treating the skin includes treating the skin by irradiating the skin with light energy in a variety of ways, such as inducing a synthesis of active substances in the skin, promoting an immune mechanism in the skin, or sterilizing pathogens on the skin.

The first light source 30 emits first light of various wavelength bands, and is provided in singular or plural.

In an example embodiment of the present disclosure, the first light may be a light of a wavelength band corresponding to at least one of infrared light, visible light, and ultraviolet light.

The first light may be a light having a wavelength band corresponding to a red visible light to a near infrared light. The first light may correspond to a light in a wavelength band of about 610 nm to about 940 nm. In an example embodiment of the present disclosure, the first light may be a light in the wavelength band corresponding to the red visible light, for example, from about 610 nm to about 750 nm, or may be a light in a wavelength band corresponding to an infrared light, for example, from about 750 nm to about 940 nm. Alternatively, in an example embodiment of the present disclosure, the first light may be a light of about 830 nm, 850 nm, or 890 nm in the wavelength band corresponding to the infrared light.

When the wavelength band corresponding to the red visible light to near infrared light is applied to the skin, blood vessels are expanded and blood circulation is promoted. That is, the first light improves blood flow, and as a result, promotes immune action.

In detail, the red visible light to near-infrared light act on the skin of the subject to be treated and intracellular mitochondria are stimulated to generate adenosine tri-phosphate (ATP), reactive oxygen species (ROS), and/or nitrogen oxide (NO). The ATP, ROS, and/or NO act on a wounded area to promote healing of wound. The ATP and ROS induce expression of genes involved in an inflammatory response, which is an immune response required for wound healing, and genes needed for cell growth. Thus, the inflammatory response and cell growth are induced in a damaged tissue part, resulting in the healing of the wound. The NO promotes migration of immune cells and increases a supply of oxygen and nutrients to accelerate a tissue healing process. In addition, the NO expands capillaries of a surrounding tissue and induces formation of new capillaries.

In an example embodiment of the present disclosure, the first light may be a light of a wavelength band corresponding to a blue among the visible light. The first light may correspond to a light in a wavelength band of about 400 nm to about 500 nm. In an example embodiment of the present disclosure, the first light may be a light in the wavelength band of about 400 nm to about 420 nm. In an example embodiment of the present disclosure, more specifically, the first light may be a light having a wavelength of 405 nm.

According to an example embodiment of the present disclosure, the first light may correspond to a wavelength band of about 400 nm to about 500 nm, but it may be light except for a wavelength band of about 435 nm to about 440 nm. When the light of the wavelength band of about 435 nm to about 440 nm is exposed to the human skin for a while to constantly expose the excessive light of the blue wavelength to the human body, risks of developing eye diseases such as macular degeneration and cataract may be increased.

When the blue light is provided to the skin as the first light, the blue light may kill bacteria present on or in the skin. The blue light corresponds to an absorption wavelength of a porphyrin present in bacteria. When the blue light is applied to the bacteria, the porphyrin in the bacteria absorbs the blue light and reactive oxygen species are generated in cells of the bacteria by energy of the blue light. The reactive oxygen accumulates in the cells of bacteria to oxidize cell walls of the bacteria, and as a result, the bacteria are killed.

In an example embodiment of the present disclosure, the first light may have the visible light spectrum similar to sunlight in the form of evenly mixed light of the entire wavelength bands. However, the first light according to an example embodiment of the present disclosure may be different from the sunlight in that the first light does not emit the ultraviolet wavelength band. The first light according to an example embodiment of the present disclosure emits a light having a wavelength band of about 380 nm to about 780 nm substantially corresponding to the entire wavelength band of the visible light.

In an example embodiment of the present disclosure, a term “similar to sunlight” means that an overlapping area based on a normalized solar spectrum is more than a specific value in comparison with the conventional light source and a deviation of a peak from the normalized solar spectrum (a deviation degree from the peak of the normalized solar spectrum) is lower than a specific value. For example, in an example embodiment of the present disclosure, the first light source 30 may emit a light having an area of about 55% or more of an area of the normalized solar spectrum and a peak of the first light may have a deviation of about 0.14 or less from the normalized solar spectrum. The normalized solar spectrum may be represented by Equation 1 below.

$$E ( λ , T ) = \frac{2 h {c}^{2}}{{λ}^{5}} \cdot \frac{1}{{e}^{hc / λ kT} - 1} \qquad Equation 1$$

where: λ: wavelength (um)

h: Planck's constant

c: speed of light

T: absolute temperature

k: Boltzmann constant

The first light may have the spectrum similar to the sunlight to have an effect similar to an effect of frequent exposure to the sunlight, and therefore, synthesis of vitamin D may be facilitated or prevalence of diseases such as myopia may be lowered.

In an example embodiment of the present disclosure, the first light may be a light in the ultraviolet wavelength band. When the first light is the light in the ultraviolet wavelength band, the first light has an effect of sterilizing bacteria which are penetrated on or in the skin. The first light may be a light in a wavelength band of about 100 nm to about 400 nm and may be UVA, UVB, or UVC. UVA may have a wavelength band of about 315 nm to about 400 nm, UVB may have a wavelength band of about 280 nm to about 315 nm, and UVC may have a wavelength band of about 100 nm to about 280 nm. In an example embodiment of the present disclosure, the first light may correspond to UVC and may have a wavelength band of about 240 nm to about 280 nm. In an example embodiment of the present disclosure, more specifically, the first light may be a light having a wavelength of 275 nm.

When the first light is the ultraviolet light, the first light may modify a structure of DNA present in the bacteria to perform sterilization. When the first light is applied to bacteria, the DNA in bacteria absorbs the first light and a change in the DNA structure occurs by energy of the first light. In particular, a binding of thymine and adenine in the DNA is broken by absorption of the light, and, because bases constituting the DNA, such as purine and pyrimidine, strongly absorb the applied ultraviolet light, the absorption of the light results in formation of thymine dimers. This process leads to modification of the DNA, which leads to death of bacteria because the modified DNA is incapable of cell proliferation. The DNA may absorb a light in the wavelength range of about 240 nm to about 280 nm.

Here, when the first light is the ultraviolet light, the ultraviolet light may correspond to at least one wavelength band of UVA, UVB, and UVC wavelength bands. Furthermore, when a predetermined dose of the first light applied to the human body in a harmless range per day is referred to as an allowable dose, the first light may be irradiated to the skin within the allowable dose. For example, the first light may be irradiated to the skin at a dose of about 30 J/m² to about 10,000 J/m².

In an example embodiment of the present disclosure, it is described that the first light corresponds to the ultraviolet band, visible band, or infrared wavelength band, respectively, but it is not limited thereto, and the wavelength is sufficient as long as the light is capable of treating the skin. The first light may also be a combination of lights in at least two wavelength bands of ultraviolet, visible, and infrared wavelength bands.

The erythema detector 40 is for detecting occurrence of skin erythema, which may occur when the first light is excessively irradiated to the skin.

Erythema reaction is one of skin reactions which appear on the skin when the first light is irradiated to the skin with a dose greater than or equal to the allowable dose, and refers to a phenomenon in which blood flow increases due to expansion of blood vessels in dermis and the skin turns red. The erythema reaction may differ in occurrence of erythema, time taken to develop erythema, degree of reaction, depending on a type of the first light. When the first light is the ultraviolet light, the erythema reaction may occur with a smaller dose than doses applied with light of other wavelength bands, for example, the visible light or the infrared light. In particular, the erythema reaction may easily occur when exposed to UVB in the ultraviolet light band. However, the erythema reaction may also occur in a light of other wavelengths, and when the first light is the visible light or the infrared light, the erythema reaction may occur when exposed to a larger amount of dose than that of the ultraviolet light for a while. In addition, a delayed erythema reaction may occur when the skin is exposed to the ultraviolet rays, and, in this case, erythema may occur after 2 to 6 hours of continuous exposure to the ultraviolet rays, and erythema may be most severe after 24 hours. After 3 to 5 days, erythema subsides due to deposition, and erythema gradually disappears over time.

As shown in FIGS. 1-2, the erythema detector 40 may include the second light source 41 which emits a second light in the visible wavelength band to the skin and at least one sensor 43 which receives the second light traveling through the skin to detect erythema.

One or more second light sources 41 are provided and the second light corresponds to the light of the visible light wavelength band which is capable of representing a color corresponding to a color space. For example, the second light may be a light similar to CIE standard light (D65) and high color rendering (CRI 90 or higher) sunlight. The sensor 43 senses a light in which the second light is reflected, scattered, or distributed by the skin to provide what is quantifiable in the color space. Here, the color space may be CIE XYZ or CIE L*a*b*. CIE L*a*b* is a color value defined by the International Lighting Commission (CIE) and is a coordinate expressing color difference and color space which our eyes are capable of recognizing. Here L* represents lightness, a* relates to red-green, in which a positive value represents red and a negative value represents green, and b* relates to yellow-blue in which a positive value represents yellow and a negative value represents blue.

The second light source 41 may provide the second light to the skin in various forms to allow the second light to go toward the sensor 43 after being scattered, reflected, and distributed. For example, the second light source 41 may be disposed to allow an incident angle of the second light to be about 45 degrees with respect to the skin.

The sensor 43 may sense the second light emitted from the second light source 41, and may be a color sensor such as a photodiode. In addition, the sensor 43 may be an image sensor camera, such as a charge-coupled device (CCD) or a complementary metal oxide semiconductor image sensor (CMOS). The sensor 43 receives the second light, which passes through the skin, to obtain color information of the second light received by the sensor 43. The color information may be color coordinate values in the color space, for example, L*, a*, and b* values in CIE L*a*b*.

The controller 50 receives the color information detected by the sensor 43 in the erythema detector 40 and determines whether erythema is generated based on the color information. When the controller 50 determines that erythema occurs, the controller 50 may turn off the first light source 30, and when the controller 50 determines that no erythema occurs, the controller 50 may maintain irradiation of the first light source 30.

The controller 50 may control whether light is emitted from the first and second light sources 30 and 41, whether the sensor 43 is operated, the amount of the light, intensity of the light, emission time, and detection time of the sensor 43. The power supplier 60 is electrically connected to the controller 50 to supply power to the first and second light sources 30 and 41 and the controller 50. In FIG. 2, although the power supplier 60 supplies power to the first and second light sources 30 and 41 through the controller 50, the present disclosure is not limited thereto, and the power supplier 60 may be directly connected to the first and second light sources 30 and 41 to supply power to the first and second light sources 30 and 41.

After receiving the color information on the second light propagating through the skin, the controller 50 determines a normal state and an erythema occurrence state based on the color information, which will be described later.

In the present embodiment, the controller 50 drives the first light source 30 and the erythema detector 40 simultaneously or separately. The first and second light sources 30 and 41 may be turned on and off simultaneously, and each of the first and second light sources 30 and 41 may be turned on and off separately. In an example embodiment of the present disclosure, after the first light source 30 is turned on, the erythema detector 40 may be turned on at a specific interval and the second light source 41 may work when the erythema detector 40 is turned on.

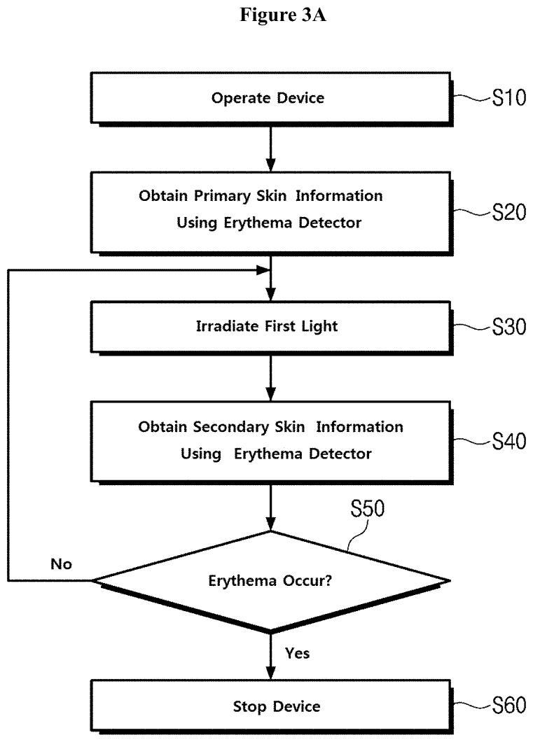

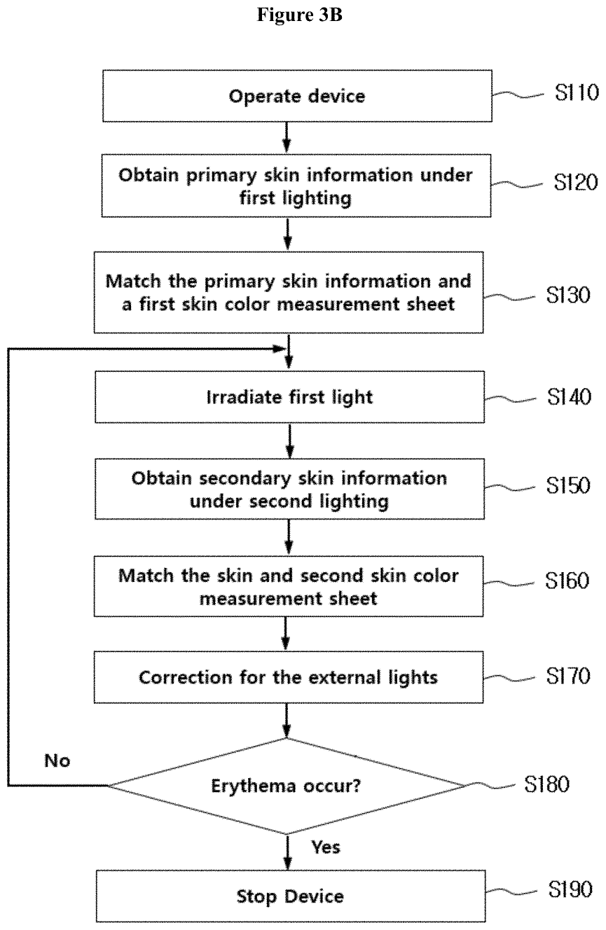

FIG. 3A is a flowchart illustrating an operation sequence of a light irradiation device according to an example embodiment of the present disclosure.

Referring to FIG. 3A, first, the light irradiation device starts operation in S10. Power is supplied to the first light source, the erythema detector, and the controller through the power supplier to perform the light irradiation device.

The operation of the light irradiation device may be manually performed by a user, directly, but it is not limited thereto. The operation of the light irradiation device may be automatically performed at a specific time through pre-programming.

Next, primary skin information about a condition of the skin is obtained using the erythema detector in S20. A skin to be checked, i.e., a target skin, may be determined, information of a portion of the target skin may be captured, and color coordinate values may be obtained from the captured information such as the captured image to obtain the primary skin information. Here, the primary skin information may refer to skin information before driving the first light source, that is, before the first light is irradiated to the skin, and in detail, may be color information in the color space of the skin.

Next, the first light source is turned on and the first light is irradiated to the skin in S30. The first light may be provided to the skin continuously, or discontinuously and periodically. For example, when the first light is the ultraviolet light, the ultraviolet light may be provided to the target skin a plurality of times for a relatively short time in consideration of the allowable dose of the human body, and when the first light is the infrared light, the infrared light may be provided continuously for a relatively long time.

Hereafter, the erythema detector is turned on to obtain secondary skin information in S40. The target skin may be determined, information of a portion of the skin may be captured, and color coordinate values may be obtained from the captured information such as the captured image to obtain the secondary skin information. Here, the secondary skin information may refer to skin information after applying the first light to the skin by driving the first light source or during applying the first light to the skin, and specifically, the secondary skin information may be color information in the color space of the skin to which the first light is applied.

In an example embodiment of the present disclosure, the primary skin information and the secondary skin information may be obtained automatically or manually. In the case of the primary skin information, the erythema detector may be set to be automatically executed along with the operation of the device, or the user may directly execute the operation of the erythema detector after setting to be executed manually.

In an example embodiment of the present disclosure, when obtaining the primary skin information it may be set to obtain the primary skin information before each irradiation of the first light, but it is not limited thereto. For example, when the device is limited to a specific user or there is not much change in the external environment, the primary skin information may be obtained once for the first time to be stored, and then each time the first light is irradiated, the primary skin information on the stored user may be retrieved and used.

The secondary skin information may be obtained continuously in real time, but it may also be obtained periodically at regular time intervals. For example, while the irradiation of the first light source is continued, the second light source is continuously turned on together, and thus the sensor may obtain secondary skin information in real time. Alternatively, the irradiation of the first light source may be continued, but the second light source may be periodically turned on, for example, once every 10 minutes, once every hour, etc., at which time the sensor is activated to obtain secondary skin information.

After the controller acquires both primary skin information (e.g. L*, a*, and b* values) and secondary skin information from the erythema detector, the primary skin information is compared with the secondary skin information to check whether erythema occurs in S50. In the present embodiment, the controller may include a comparator 51 for comparing the primary skin information and the secondary skin information, and the comparator 51 may compare L*, a*, and b* values in the primary skin information and those values in the secondary skin information, respectively. The controller may determine changes of L*, a*, and b* value through the comparison in the comparator 51, and it may be determined that erythema occurs when the amount of change of L*, a*, and b* values exceeds a specific value (i.e., the allowable value). For example, in the skin erythema, L* value decreases and a* value increases with skin color.

Here, when information on the skin color of the user is subdivided to various degrees, accuracy may be improved. For example, after subdividing the information on the skin color into five or ten or more levels, the allowable amount of change may be set based on the subdivided values. The allowable values vary depending on the type of skin or condition of the skin.

Upon determination by the controller that erythema has occurred, the device stops operation in S60. That is, irradiation of the first light is blocked by turning off the first light source. Upon determination by the controller that erythema has not occurred, irradiation of the first light is continued.

The controller calculates a change rate of skin color, and determines that erythema occurs when the change rate of skin color exceeds a specific range (the allowed value).

The change rate of the skin color determined to have erythema may be predetermined in consideration of race, skin color, skin type, and the like and may be stored in the controller.

An operation mechanism of the light irradiation device according to an example embodiment of the present disclosure is described below. First, a case where an external lighting is the same in obtaining of the primary skin information and the obtaining of the secondary skin information will be described.

First, a target of the skin to be treated is selected, information of a part of the skin corresponding to the target is captured, and then the primary skin information of the target is obtained.

After obtaining primary skin information, the secondary skin information is obtained after applying the first light or during applying the first light. The secondary skin information is obtained by recapturing the skin portion from which the primary skin information is obtained, and then extracting the color information of the captured portion.

When the skin color change rate of the extracted color information coincides with the primary skin information or is within a specific range, it is determined that erythema has not occurred, and when the skin color change rate is not within the specific range, unlike the primary skin information, it is determined that erythema has occurred.

When it is determined that erythema occurs, the device stops operation to suspend the irradiation of the first light.

Next, a case where the external lighting for the obtaining of the primary skin information is different from that for the obtaining of the secondary skin information will be described. For example, the external lighting may be a fluorescent light or an LED light, and the same skin may be determined to be different colors depending on the external lighting. When the same skin color is determined as a different color depending on the external lighting, the occurrence of erythema may not be accurately determined.

In the present embodiment, different external lightings may assume first and second lightings, respectively, and measurement sheets which assume the color change as the first and second lightings themselves may be prepared to correct the change of the color in advance.

FIG. 3B is a flowchart illustrating an operation sequence of a light irradiation device according to another example embodiment of the present disclosure.

First, the light irradiation device starts operation. (S110)

A target skin region to be treated is selected and information of the relevant skin is captured under the first lighting, and then primary skin information of the target skin region is obtained. (S120) Here, when information of the skin is captured and the primary skin information is obtained, a first skin color measurement sheet for the skin color corresponding to the first lighting is prepared and matched with the primary skin information. (S130)

Here, the first skin color measurement sheet may be pre-manufactured with a plurality of colors, for example, a second skin color measurement sheet, in different colors depending on a type of lighting, considering that the same skin is seen in a different color depending on a light source. Here, the measurement sheet may be a physically existing object but it may be color information virtually existing on the controller.

After obtaining the primary skin information, the secondary skin information is obtained. (S150) The secondary skin information is obtained under a second lighting after applying the first light or during application of the first light. (S140) The secondary skin information is obtained by re-capturing the relevant skin portion from which the primary skin information is obtained, and then extracting color information of the captured portion.

Here, when capturing the skin and obtaining the secondary skin information, the second measurement sheet prepared in advance, which corresponds to the measurement sheet relevant to the first lighting, is matched to the skin. (S160)

The first measurement sheet and the second measurement sheet may be preset in correspondence with the external lightings, and the second measurement sheet may be corrected to what extent the color of the second measurement sheet is changed based on the first measurement sheet. (S170)

After the correction for the external lights is pre-performed, when the skin color change rate of the color information extracted from the actual user's skin coincides with the primary skin information or within a specific range, it is determined that erythema does not occur, and when the skin color change rate is not within the specific range, unlike the primary skin information, it is determined that erythema occurs. (S180)

When it is determined that erythema occurs, the device is stopped to suspend the irradiation of the first light. (S190)

In the present embodiment, even when the skin region is captured under different external lightings, correction of a standard skin color sheet may give the same effect as measured under the same condition. This allows correct determination as to whether erythema occurs, regardless of the external lightings.

In an example embodiment of the present disclosure, when it is determined that erythema occurs, it is basic to stop the device, but a driving method of the light irradiation device is not limited thereto. For example, after stopping the device, additional tertiary skin information may be acquired to check whether erythema disappears and the first light source may be turned on again.

In an example embodiment of the present disclosure, an additional component may be further provided to further clarify whether erythema occurs. For example, erythema detector may further include a temperature sensor for measuring temperature of the skin. The temperature sensor may be an infrared sensor, or may be a contact sensor which is in direct contact with the skin to measure the temperature.

As described above, in the light irradiation device according to an example embodiment of the present disclosure, when ultraviolet light, infrared light, visible light, and the like are used for medical or cosmetic purposes, the erythema reaction may be continuously measured to stop the device before the occurrence of erythema, thereby increasing safety of treatment.

For a general light irradiation device, a doctor checks whether a skin abnormality such as erythema occurs over several days after exposing the light to the skin of a patient to check a skin type and a dosage of the patient. Thus, such determination may be time consuming and costly. In addition, for a personal light irradiation device used for esthetic or treatment, occurrence of skin abnormality such as erythema may not be checked in real time. On the contrary, the light irradiation device according to an example embodiment of the present disclosure may easily check whether or not erythema occurs in real time with the application of the first light, thereby ensuring safety.

The light irradiation device according to an example embodiment of the present disclosure may be implemented in various forms for the treatment of the skin.

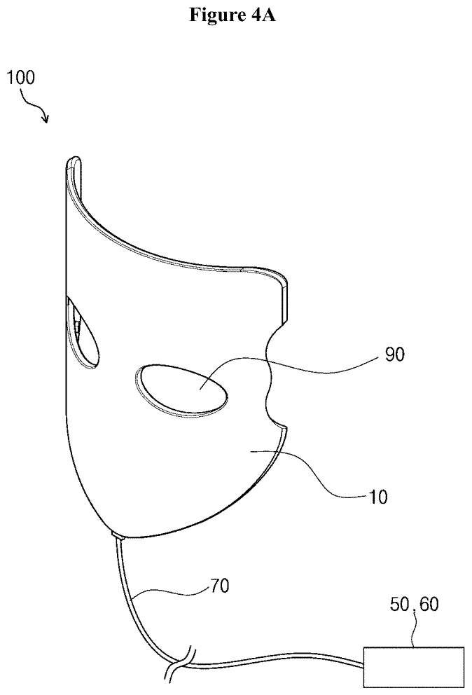

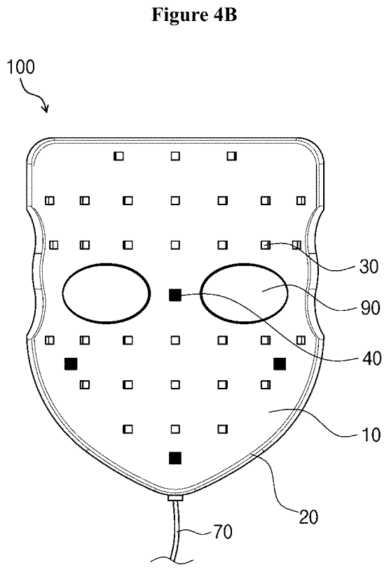

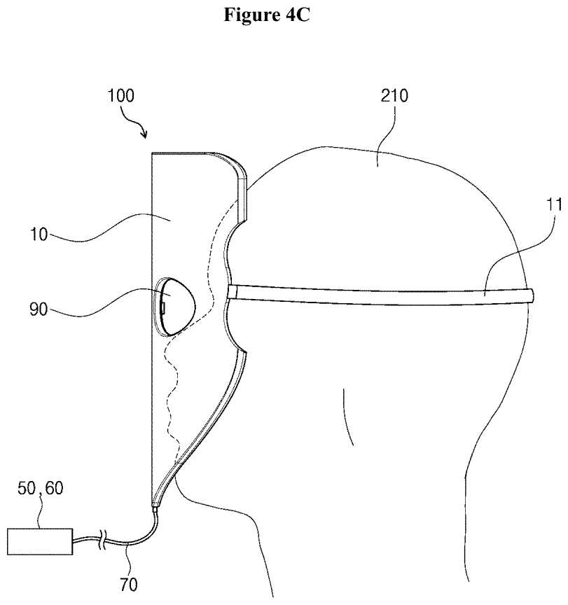

FIG. 4A is a perspective view of a light irradiation device according to an example embodiment of the present disclosure, which illustrates a light irradiation device manufactured in a form of a mask. FIG. 4B is a plan view illustrating a surface facing a face, that is, a back surface of the light irradiation device, in the light irradiation device of FIG. 4A. FIG. 4C is a side view in which the light irradiation device of FIG. 4A is worn on the face.

Referring to FIGS. 4A to 4C, the light irradiation device 100 according to an example embodiment of the present disclosure may include a main body 10, the substrate 20 provided on the main body 10, the first light source 30, and the erythema detector 40, which are provided on the substrate 20. The first light source 30 and the erythema detector 40 may be equipped together with the first light source 30 and the erythema detector 40 on the main body 10, and as shown, may be connected to the controller 50 and the power supplier 60 through a separate wire 70.

The main body 10, which forms the overall shape of a mask, may cover the whole or at least part of the face. The shape of the mask may have a shape similar to that of the face, and the shape of the mask may not be limited as long as it covers at least a part of the face even when it is different from the shape of the face. In an example embodiment of the present disclosure, a light irradiation device having a shape similar to that of the face is shown as an example.

A front surface of the main body 10 is a surface which looks to the outside and the back surface of the main body 10 is a surface facing the face. The first light source 30 and the erythema detector 40 are provided on the back surface facing the face. The first light source 30 may be provided in singular or plural and, in the present embodiment, may be provided in plural. The erythema detector 40 may also be provided in singular or plural, and in the present embodiment, may be provided in plural.

The first light sources 30 may be arranged in various forms. For example, the first light sources 30 may be arranged in a matrix shape, or may be randomly arranged. An arrangement of the first light sources 30 may vary depending on a portion requiring treatment by the first light while the first light sources 30 corresponds to the face. For example, more first light sources 30 may be provided in an area of the back surface, which corresponds to a cheek or forehead of the face, and less first light sources 30 may be provided in an area of the back surface, which corresponds to a nose or chin. The arrangement of the first light sources 30 is shown as an example and may be changed in various forms as necessary.

The erythema detector 40 may be disposed at a place where erythema frequently occurs or a place to confirm occurrence of erythema. For example, the erythema detector 40 may be changed in various arrangement portions, for example, may be disposed in an area of the back surface corresponding to the nose or in an area of the back surface corresponding to a jaw or cheek.

In the present embodiment, although the second light source and the sensor of the erythema detector 40 are shown as one component without separation, they are not limited thereto. The second light source and the sensor of the erythema detector 40 may be separated and disposed at different positions.

In an example embodiment of the present disclosure, the mask may be provided with through holes 90 for protecting eyes. Parts where the through holes 90 are formed are where the eyes are located on the face. Since the first light source 30 or the second light source is not provided in the parts where the through holes 90 are formed, the first light or the second light is prevented from being exposed to the eyes.

In an example embodiment of the present disclosure, the through-hole 90 for protecting the eyes is shown in the main body 10, but is not limited thereto. If the eye can be protected by providing a separate shade for protecting the eye, the through holes 90 may not be provided.

A fixing band 11 for fixing the mask-type light irradiation device to a head 210 of the user may be provided at one side of the main body 10. However, various types of fixing members may be used instead of the fixing band 11 as long as the light irradiation device is capable of being fixed to the head 210 of the user. For example, the light irradiation device may be manufactured in a form of a helmet, and the first light source 30 and the erythema detector 40 may be provided at an inner portion corresponding to the face.

Although not shown, various components may be further added to allow the mask-type light irradiation device to be stably worn on the face of the user. For example, a nasal pedestal may be provided on the back surface of the main body 10 or a support member protruding from the main body 10 toward the face to space a distance between the face and the main body 10 and to allow the face and the first light source 30 to be spaced apart by a specific distance.

In an example embodiment of the present disclosure, the light irradiation device may further be provided with an optical unit for selectively focusing or dispersing the light emitted from the first and second light sources. The optical unit may focus the light generated from the first and second light sources into a narrow area or a wide area as necessary. Alternatively, the light may be focused or dispersed in a uniform or non-uniform form based on a position to be irradiated with the light. The optical unit may include at least one lens as needed, and the lens may perform various functions such as focusing, dispersing, uniformizing, and non-uniformizing the light from the first and second light sources.

In an example embodiment of the present disclosure, an edge of the main body 10 may be provided with a blocking film to prevent the light emitted from the back surface of the main body 10 from going toward the outside. The blocking film may cover between the edge of the main body 10 and the face.

In the case of the mask-type light irradiation device, the region to be treated may be selected and the first light sources 30 and the erythema detector 40 disposed on a part corresponding to the selected region may be operated. The selection may be performed directly by the user or automatically.

In the case of the mask-type light irradiation device, the mask-type light irradiation device is for treating the face area and the face is often more sensitive than other areas to frequently generate erythema. In this example embodiment, the mask-type light irradiation device provides the erythema detector 40 capable of confirming whether or not erythema is generated and thus erythema is prevented in addition to the light treatment.

In an example embodiment of the present disclosure, in the above-described example embodiment, although a plurality of erythema detectors 40 are formed, the present disclosure is not limited thereto, and may be provided in singular. When a single erythema detector 40 is provided in the light irradiation device, the erythema detector 40 may be fixedly arranged at a center side but may be provided in a form capable of moving to various positions.

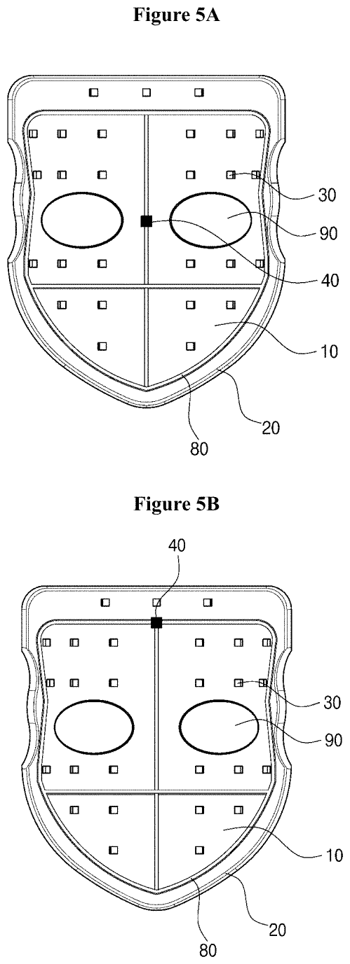

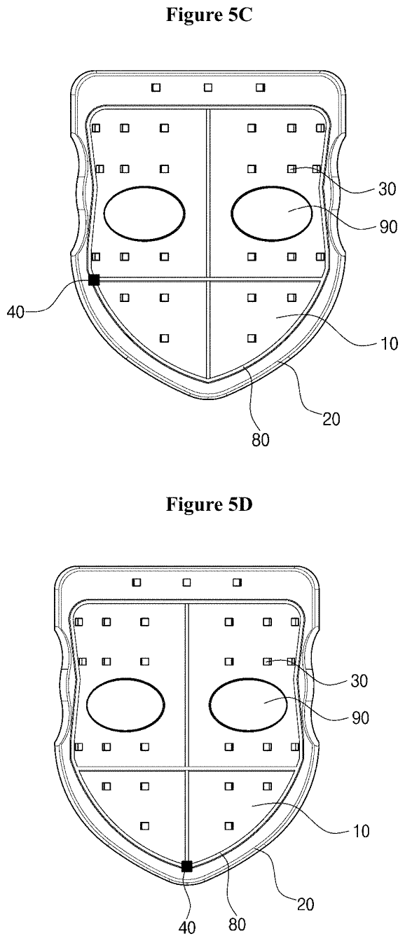

FIGS. 5A to 5D illustrates the erythema detector 40 which is movable within a light irradiation device.

Referring to FIGS. 5A to 5D, at least one movement path which enables movement of the erythema detector 40 may be provided on the back surface of the mask-type light irradiation device. The movement path may be provided in various forms, for example, may have a form of a rail 80. The erythema detector 40 may be equipped with a moving member such as a roller, and may move to various areas along the rail 80. For example, FIG. 5A illustrates that the erythema detector 40 moves to the nose, FIG. 5B illustrates that the erythema detector 40 moves to the forehead, FIG. 5C illustrates that the erythema detector 40 moves to the cheek, and FIG. 5D illustrates that the erythema detector 40 moves to the chin. When the erythema detector 40 is movable, a small number of erythema detectors 40 may determine whether erythema occurs in a wide area.

In the present embodiment, the second light source and the sensor of the erythema detector 40 are illustrated as one component, but each may be disposed separately, and, in this case, the second light source may be movable, the sensor may be movable, or both of first light source 30 and the sensor may be movable. The second light source and the sensor may move along the rail 80 and measure an erythema value of the relevant area, and thus may measure in real time or may set a specific interval (by dose) through a program to measure whether erythema occur or not. In addition, positions where the user specifically wants to check for erythema may be coordinated to be measured.

The movable erythema detector may be applied to various types of light irradiation devices even when the light irradiation device is not a mask type.

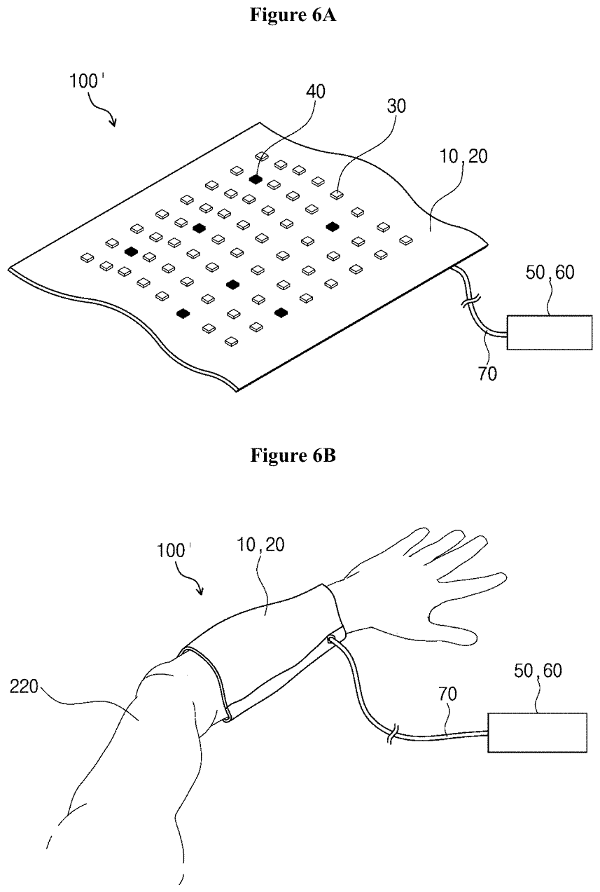

FIG. 6A is a perspective view of a light irradiation device according to an example embodiment of the present disclosure and illustrates a light irradiation device manufactured in a wearable form. FIG. 6B is a perspective view illustrating a state in which the light irradiation device of FIG. 6A is worn on a human arm 220.

Referring to FIGS. 6A and 6B, a light irradiation device according to an example embodiment of the present disclosure may include the main body 10, the substrate 20 provided on the main body 10, the first light source 30 and the erythema detector 40, which are provided on the substrate 20. In the present embodiment, the substrate 20 and the main body 10 may be formed separately to allow the substrate 20 to be placed on the main body 10, but the present disclosure is not limited thereto, and the substrate 20 and the main body 10 may be formed integrally. The substrate 20 and the main body 10 may have flexibility, and may be bent or folded due to the flexibility.

The first light source 30 and the erythema detector 40 may be mounted on the main body 10 together with the first light source 30 and the erythema detector 40, and as shown, may be connected to the controller 50 and the power supply 60 through the separate wire 70.

The light irradiation device according to the present embodiment may be modified in various forms due to its flexibility and may be wearable on the body as shown in FIG. 6B. Although the light irradiation device is worn on a part of the arm 220 in the present embodiment, the worn form or worn position is not limited thereto. The light irradiation device according to an example embodiment of the present disclosure may be worn to, for example, ankle or waist.

Here, a surface facing the skin corresponds to a surface on which the first light source 30 and the erythema detector 40 is mounted, and a spacing support member may be further provided on the main body 10 to allow a gap between the first light source 30 and the skin to be partially maintained.

The light irradiation device according to an example embodiment of the present disclosure may measure whether the skin erythema occurs in real time, but it may measure the skin type using the sensor of the erythema detector in advance, and may control the exposure amount of the first light, for example, the exposure light amount or exposure time, within the range in which erythema does not occur depending on the skin color or condition, that is, within maximum 1 MED MED means the minimal erythema dose and varies depending on skin type and condition, exposed area, and exposure level, and 1 MED corresponds to 200 J/m²=0.02 J/m² (20 mJ/cm²).

The amount of exposure of the first light within the range in which erythema does not occur may be predetermined through preliminary examination depending on the skin type. For example, because white skin and black skin may have different allowable doses for the ultraviolet light, first, the primary skin information may be obtained to determine the skin type, and then the degree of irradiation of the first light may be adjusted depending on the skin type through the program.

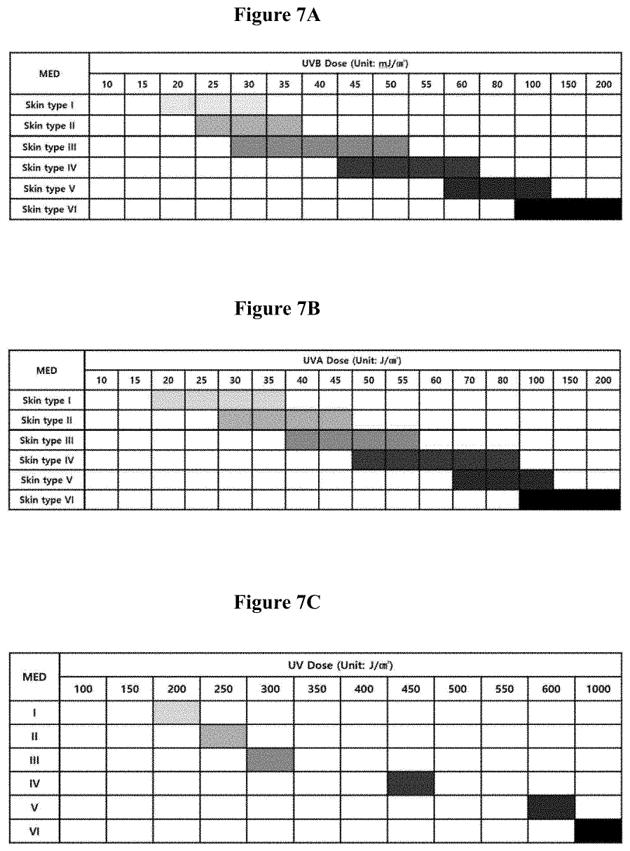

FIGS. 7A to 7C illustrate a minimal erythema dose within a range in which erythema does not occur in a first light irradiation depending on a type of skin. More specifically, FIG. 7A illustrates a case where UVB is used as the first light, FIG. 7B illustrates a case where UVA is used as the first light, and FIG. 7C illustrates a case where a combination of UVA and UVB is used as the first light.

In FIGS. 7A to 7C, skin types I to VI correspond to a method for evaluating a skin response to a ultraviolet light developed by Fitzpatrick, and is classified based on sensitivity of the skin to sunlight. Skin type I has white skin, low melanin count and high UV sensitivity. In addition, there is a high possibility of sunburn and a high risk of skin cancer. From skin type I to skin type VI, the skin color becomes darker, the number of melanin increases, and the UV sensitivity decreases. In addition, the risk of sunburn and skin cancer decreases.

Conditions for obtaining the minimum erythema dose shown in FIG. 7A are as follows.

Skin Type I/II: After UVB light is irradiated at 5 mJ/cm² or less, light irradiation is stopped and skin erythema value is measured. Repeat the process. (Maximum irradiation range I to 30 mJ/cm²/II to 35 mJ/cm²)

Skin Type III/IV: After UVB light is irradiated at 10 mJ/cm² or less, light irradiation is stopped and skin erythema value is measured. Repeat the process. (Maximum irradiation range III to 50 mJ/cm²/IV to 60 mJ/cm²)

Skin type V/VI: After UVB light irradiation 20 mJ/cm² or less, light irradiation is stopped and skin erythema value is measured. Repeat the process. (Maximum irradiation range V to 100 mJ/cm³/VI to 200 mJ/cm²)

Conditions for obtaining the minimum erythema dose shown in FIG. 7B are as follows.

Skin Type I/II: After UVA light is irradiated at 5 mJ/cm² or less, light irradiation is stopped and skin erythema value is measured. Repeat the process. (Maximum irradiation range I to 35 mJ/cm²/II to 45 mJ/cm²)

Skin Type III/IV: After UVA light is irradiated at 10 mJ/cm² or less, light irradiation is stopped and skin erythema value is measured. Repeat the process. (Maximum irradiation range III to 55 mJ/cm²/IV to 80 mJ/cm²)

Skin type V/VI: After UVA light is irradiated at 20 mJ/cm² or less, light irradiation is stopped and skin erythema value is measured. Repeat the process. (Maximum irradiation range V to 100 mJ/cm²/VI to 200 mJ/cm²)

Conditions for obtaining the minimum erythema dose shown in FIG. 7C are as follows.

Skin Type I/II: After complex light is irradiated at 50 mJ/cm² or less, light irradiation is stopped and skin erythema value is measured. Repeat the process. (Maximum irradiation range I to 200 mJ/cm²/II to 250 mJ/cm²)

Skin Type III/IV: After complex light is irradiated at 50 mJ/cm2 or less, light irradiation is stopped and skin erythema value is measured. Repeat the process. (Maximum irradiation range III to 300 mJ/cm²/IV to 450 mJ/cm²)

Skin Type V/VI: After complex light is irradiated at 100 mJ/cm² or less, light irradiation is stopped and skin erythema value is measured. Repeat the process. (Maximum irradiation range V to 600 mJ/cm²/VI to 1000 mJ/cm²)

As described above, the light irradiation device according to an example embodiment of the present disclosure may easily change the set value based on the condition desired by the user.

The light irradiation device of the present disclosure may applied to public facilities, public use spaces, and public use products to be used for public treatment or may be applied to personal facilities, personal use spaces, and personal use products to be used for personal treatment.

In addition, the light irradiation device may used as a stand-alone device. The light irradiation device may be added to another treatment device to be used.

While the inventive concept has been described with reference to exemplary embodiments thereof, it will be apparent to those of ordinary skill in the art that various changes and modifications may be made thereto without departing from the spirit and scope of the inventive concept as set forth in the following claims.

Therefore, the technical scope of the inventive concept should not be limited to the contents described in the detailed description of the specification but should be defined by the claims.

1. A light irradiation device comprising:
at least one first light source configured to emit first light of a wavelength band for treating a target skin;
an erythema detector configured to obtain color information of the target skin; and
a controller configured to:
determine whether erythema of the target skin has occurred based on the color information and
control driving of the first light source depending on occurrence of erythema, wherein the erythema detector further includes:
at least one second light source configured to emit second light to the target skin; and
at least one sensor configured to receive the second light passing through the target skin and provide the color information of the target skin.

2. The light irradiation device of claim 1, wherein the second light is light corresponding to a wavelength band of visible light.

3. The light irradiation device of claim 1, wherein the sensor detects the second light reflected, scattered, or dispersed by the target skin.

4. The light irradiation device of claim 3, wherein the controller further includes a comparator which compares between a skin color of the target skin detected before the first light is applied and a skin color of the target skin detected after the first light is applied and derives a change rate in the skin color of the target skin, the change rate indicative of whether erythema has occurred or not.

5. The light irradiation device of claim 4, wherein the skin color is represented by color coordinate values in a CIE LAB color space.

6. The light irradiation device of claim 4, wherein the controller is further configured to:
pre-set virtual skin color measurement sheets depending on a type of external lighting,
additionally correct a value due to a difference in the skin color measurement sheets due to the difference in the type of external lighting, and
then derive and compare the change rate between the skin color of the skin detected before the first light is applied and the skin color of the skin detected after the first light is applied, to determine whether erythema occurs or not.

7. The light irradiation device of claim 4, wherein the controller further includes the comparator comparing the change rate between a predetermined skin color and a skin color of the skin detected from the sensor to determine whether erythema has occurred or not.

8. The light irradiation device of claim 1, wherein the sensor includes a CCD, a CMOS image sensor, or a photodiode.

9. The light irradiation device of claim 1, wherein the erythema detector further includes a temperature sensor which measures a temperature of the skin.

10. The light irradiation device of claim 9, wherein the temperature sensor includes an infrared sensor.

11. The light irradiation device of claim 9, wherein the temperature sensor includes a contact sensor which is directly contact with the skin to measure the temperature of the skin.

12. The light irradiation device of claim 1, further comprising:
a main body on which the first light source and the erythema detector are mounted, and
wherein the main body has flexibility.

13. The light irradiation device of claim 12, wherein the first light is applied to a first region of the target skin and at least one of the second light source or the sensor is movable in the first region.

14. The light irradiation device of claim 13, wherein the main body includes a rail provided on a surface facing the skin and along a movement path of at least one of the second light source and the sensor.

15. The light irradiation device of claim 1, wherein the first light is a light of a blue wavelength band.

16. The light irradiation device of claim 1, wherein the first light is a light of a red to infrared wavelength band.

17. The light irradiation device of claim 1, wherein the first light is a light of an ultraviolet wavelength band.

18. The light irradiation device of claim 1, wherein the first light is a light in which at least two wavelength bands of ultraviolet, visible and infrared wavelength bands are combined.

19. The light irradiation device of claim 1, wherein the second light source has a wavelength band of about 380 nm to about 780 nm, has an area of about 55% or more of an area of a normalized solar spectrum within a range of about 2,600K to about 7,000K, and normalized solar spectrum is represented by the following
$$E ( λ , T ) = \frac{2 h {c}^{2}}{{λ}^{5}} \cdot \frac{1}{{e}^{hc / λ kT} - 1} \qquad Equation 1$$
where λ is wavelength (um);
h is Planck's constant;
c is speed of light;
T is absolute temperature; and
k is Boltzmann constant.
